(19) United States
(12) Patent Application Publication
(10) Pub. No.: US 2026/0207371 A1
(43) Pub. Date: Jul. 23, 2026

(54) Individual Head Forward Flexion Movement Support Apparatus

(71) Applicant: Richard D. Hughes, Centennial, CO (US)

(72) Inventor: Richard D. Hughes, Centennial, CO (US)

(21) Appl. No.: 19/031,171

(22) Filed: Jan. 17, 2025

(51) Int. Cl. A61F 5/37

(52) U.S. Cl. CPC A61F 5/3707

(57) ABSTRACT

An individual’s head forward flexion movement support apparatus that includes a planar shaped beam with first and second end portions, the beam also having a primary and a secondary side portion, the primary side portion having a primary concave arcuate shape forming an open primary channel, also a perpendicularly oriented secondary concave arcuate shape portion. Wherein operationally, the beam first end portion planar primary side portion open primary channel is adapted to cradle a chin and a forward lower jaw of the individual’s head, the beam second end portion planar primary side portion open primary channel is adapted to rest against a manubrium resulting in the support apparatus supporting the individual’s head to limit forward flexion head movement in relation to the manubrium.

### CROSS-REFERENCE TO RELATED APPLICATIONS

There are no related Patent Applications.

### BACKGROUND OF INVENTION

The present invention generally relates to head supports and more particularly to a head support that that conveniently mounts on a manubrium portion of a sternum of an individual and further supports the individual’s chin all in a self-contained manner.

One particular instance of use for the present invention is in economy air travel being especially for long international flights of 6 hours up to 20 hours or so per flight leg wherein it would be highly desirable to be able to sleep for periods in an airline seat, and even though an airline seat can recline somewhat, it is not really that much, plus even with a small airline pillow available, an individual cannot really sleep on their side and it often left with their head leaning forward, this as opposed to sleeping in a bed horizontally where the neck on a pillow can actually fully relax, trying to do this is a somewhat upright position leaves the neck in a stressed head supporting position thus precluding much real sleeping. Other situations of a similar nature would include boats, buses, trains, cars, or any additional position that requires someone to remain in a seat for extended periods and would like to sleep for a portion of that period in the seat.

One solution that is somewhat popular is the horseshoe or collar pillow, which can surround the neck like a donut giving an almost omnidirectional head support and neck support, however, there are drawbacks to the special pillow being; its bulky to carry on a plane, where space for anything extra is already cramped, it generally doesn’t provide for very firm support thus still leaving the neck stressed to support the head, plus tends to irritate the outer skin surface of the neck – somewhat light a light choking feel, that further interferes with sleep. There has been an inflatable version of the horseshoe collar pillow that while reducing initial bulk to carry and t1ravel with has the added burden of having to be inflated and deflated with every trip leg and further with the air providing a bouncy non-comfortable cushion. Sometimes these air inflatable horseshoe collar pillows are used on a tray table to rest the individual’s head on, somewhat like a massage table for laying face down with an opening for the mouth and nose, again, there have not been proven popular as the pressure on the forehead gets uncomfortable quickly and breathing is difficult, as opposed to a massage table that has an opening to breathe through, the tray table does not have an opening to breathe through, and further the tray table usually moves when the person in the seat in front of the individual reclines or un-reclines their seat back making the tray table pillow impractical, and of course in addition to airplane meal service making the tray table unavailable, plus the lateral seat mates needing to have their food and drink trays passed over the individual’s sleeping head, so all in all the tray table is not a viable solution for sleeping.

Side wing supports have been yet another option on each side of the individual’s head that are adjacent to the airline seat head rest on each side being in essence an enhanced head rest, however, again not providing forward head support and having the same bulkiness drawbacks of the horseshoe collar pillow.

Looking at the prior art in this area starting with United States Patent Number 9,283,878 to Zaouk et al., disclosed is a supporting device to enhance user comfort in a seated position that includes a hinge and straight edged sides that are disposed around the middle of a user’s back that upwardly terminate in a headrest type shape that is affixed to a flexible band that wraps about the user’s head, being particularly the user’s forehead area see FIGS. 4 and 5. Also, in another embodiment to Zaouk in looking at FIGS. 6 and 7 is a clamshell type arrangement with planar padded pieces that have a somewhat centrally located hinge (can be multiple hinges) that can hold an angle, that only has teachings for a series of sandwiched tines compressed together with a cam lever bolt, similar to what is used on a bicycle axle to frame lock. Zaouk’s teachings are limited to rigid sections joined by hinges.

Further in United States Patent Number 6,266,825 to Floyd disclosed is a traveler’s head support using a band around the top of the head that also encircles the seatback. Floyd has the major drawback of having a head band type support that will unduly put pressure on the individual’s forehead causing discomfort, in addition to the inconvenience of having to attach to the airline seat back of which could be difficult as there are numerous size, style, and configurations of airline seats to contend with. Another problem with Floyd is having to deal with different shape foreheads, as some people have foreheads that are not flat but are inclined rearward at the top causing the head strap to slide upward and off and not give any head support, plus if the individual has eye glasses they could interfere with the head strap.

Continuing in the applicable prior art in United States Patent Number 8,985,693 to Purpura et al., disclosed is a transport vehicle upright sleep support system that tries through a complicated attachment to an airline seat, a massage table type frontal face rest with an aperture opening for breathing while the individual rests their forehead, checks, and chin against the outer periphery of the aperture. The seat attachment includes two extension members that are disposed on each side of the user’s neck for support of the quasi massage table face support from the airline seat back, wherein the extension members could cause neck skin irritation, further the complicated airline seat attachment and the overall high bulk of the Purpura system seem to be formidable obstacles to overcome.

Further, in the applicable prior art in United States Patent Number 8,393,017 to Sheren, disclosed is a spinal injury prevention apparatus. Not really related to the airline travel sleeping assistance product, however, being relevant in that Sheren in a neck collar that rests against the chest, the tops of the shoulders, and the upper back of an individual using the neck support collar, however, requiring for head support in all directions the use of a full face helmet by the individual, noting that this type of collar is of necessity of a rigid construction, having the distinct advantage of a wide berth of room around the user’s neck, so that there is virtually no contact of the collar onto the user’s neck outer skin surface resulting in comfort and flexibility in the user being able to freely rotate and tilt their head to a limited extent.

Next, in the applicable prior art in United States Patent Application Publication Number 2013/0333708 to Hassan, discloses a chin and neck support device that includes an extension element disposed between the chest and chin, wherein the extension element is additionally supported by a band that is positioned about the extension element and then around the back of the user’s neck in order to more securely hold the extension element in place. In Hassan, the extension element is wedged between the chin and chest thus effectively keeping the user’s mouth pushed shut, however, not interfering with the user’s swallowing or beathing. The purpose of Hassan is to help prevent snoring or sleep apnea by preventing relaxation of the lower jaw, base of the tongue, and neck, wherein Hassan could be used in an airline seat for sleeping. Hassan does take some amount of work to get the apparatus attached and adjusted properly and could cause back of neck irritation due to the band’s pressure on the back of the neck.

Moving onward, in the applicable prior art in United States Patent Application Publication Number 2010/0168628 to Hopfenspirger, disclosed is a head flexion support device that is essentially an arcuate element that is placed upon the chin and chest, wherein the inward arc does not come into contact with the user’s neck outer skin surface with a support band placed about the back of the user’s neck and over the outer or concave side of the arcuate element to hold the arcuate element against the chin and chest on opposing ends, however, with the support band putting pressure on the back of the user’s neck to hold the arcuate element in place, similar to Hassan.

What is needed is a small, compact, and customizable head support to specifically support head forward flexion movement that is easy to setup and takedown specifically benefitting the airline traveler, due to the ever-increasing limitations upon carry-on bags for weight and size by the common airline carriers, any additional bulk and weight that these seat comfort sleeping solutions provide must be absolutely minimal. Further, a consideration of the convenience of setting up the seat sleep comfort device needs to be considered, i.e. if it needs manual inflating / deflating to stow and take with you-this is an inconvenience to consider, plus is the seating sleeping comfort solution bulky and or heavy-if so, this could be a major drawback, because travelling “light” these days is more important than ever, especially with the previously mentioned carrier restrictions on carry-on items. In addition, for the seat sleeping comfort device, if a number of attachment straps / buckles are required, this further adds to the inconvenience, as the seat sleeping comfort device must be deployed and taken down for each flight leg of the traveler’s trip, making for multiple setups and takedowns, adding extra time and hassle. Also, another consideration is safety, as when the traveler is using the seat sleep comfort device, in case of an emergency, i.e. wherein the traveler would have to quickly exit their airline seat, the seat sleep comfort device cannot impede the traveler’s exit from the airline seat in any manner, as the traveler must be able to leave their airline seat and the airline seat sleep comfort device without having to take apart of disengage anything related to the airline seat sleep comfort device. Thus, it is imperative that the passenger seating sleep comfort device be of absolutely minimal size and weight to be a minimal inconvenience to the user to take with them and minimal effort to set up and take down on their travels.

### SUMMARY OF THE INVENTION

Broadly, the present invention is an individual’s head forward flexion movement support apparatus, wherein the individual’s head includes a chin and a forward lower jaw portion, the individual further having a manubrium portion of a sternum upon a chest of the individual, the support apparatus includes a planar shape beam having a first end portion and an opposing second end portion, with a beam mid-portion disposed therebetween the beam first and second end portions. The beam having a longitudinal axis spanning therebetween the beam first and second end portions, further the beam including a crosswise axis positioned perpendicular to the longitudinal axis, the crosswise axis is also parallel to the planar portion of the beam, also a shortwise axis is positioned perpendicular to both the longitudinal axis and the planar portion of the beam.

The individual’s head forward flexion movement support apparatus planar shape having a primary side portion and an opposing secondary side portion, the planar shape primary side portion having a primary concave arcuate shape parallel to the crosswise axis, the primary concave arcuate shape forming an open primary channel along the longitudinal axis from the first end portion to the beam mid-portion and onto the beam opposing second end portion. The planar shape secondary side portion having a secondary concave arcuate shape parallel to the longitudinal axis encompassing the beam mid-portion. Wherein operationally the beam first end portion planar primary side portion open primary channel is adapted to cradle the chin and forward lower jaw of the users head, the beam second end portion planar primary side portion open primary channel is adapted to rest against the manubrium resulting in the support apparatus supporting the individual’s head to limit forward flexion head movement in relation to the manubrium.

These and other objects of the present invention will become more readily appreciated and understood from a consideration of the following detailed description of the exemplary embodiments of the present invention when taken together with the accompanying drawings, in which;

### BRIEF DESCRIPTION OF DRAWINGS

FIG. 1 shows an upper perspective view of the individual’s head forward flexion movement support apparatus that includes the planar shaped beam that further has the first end portion, the mid portion, and the second end portion, with the longitudinal axis spanning therebetween, also with the primary and secondary concave arcuate shapes shown;

FIG. 2 is a front elevation view of the individual’s head forward flexion movement support apparatus that includes the planar shaped beam that further has the first end portion, the mid portion, and the second end portion, with the longitudinal axis spanning therebetween, also with the primary concave arcuate shape shown;

FIG. 3 is a side elevation view of the individual’s head forward flexion movement support apparatus that includes the planar shaped beam that further has the first end portion, the mid portion, and the second end portion, with the longitudinal axis spanning therebetween, also with the secondary concave arcuate shape shown;

FIG. 4 is a top view of the individual’s head forward flexion movement support apparatus that includes the planar shaped beam that further has the first end portion, the mid portion, and the second end portion, with the longitudinal axis spanning therebetween, also with the primary concave arcuate shape shown;

FIG. 5 is a rear view of the individual’s head forward flexion movement support apparatus that includes the planar shaped beam that further has the first end portion, the mid portion, and the second end portion, with the longitudinal axis spanning therebetween;

FIG. 6 is a bottom view of the individual’s head forward flexion movement support apparatus that includes the planar shaped beam that further has the first end portion, the mid portion, and the second end portion, with the longitudinal axis spanning therebetween, also with the primary concave arcuate shape shown;

FIG. 7 is cross section cut 7-7 positioned in two places from FIG. 1, wherein FIG. 7 shows a semi-rigid malleable core encompassed by a resilient outer wrap to form the planar shaped beam;

FIG. 8 is an in use view shown as a side elevation view of the individual’s head forward flexion movement support apparatus that includes the planar shaped beam that further has the first end portion, the mid portion, and the second end portion, with the longitudinal axis spanning therebetween, also with the secondary concave arcuate shape shown, wherein the second end portion of the planar beam open primary channel that is adapted to rest against the manubrium of the individual’s sternum and further the first end portion of the planar beam that through the open primary channel cradles the chin and forward lower jaw of the individual providing head forward movement limitation flexion support relative to the individual’s sternum;

FIG. 9 is cross section cut 9-9 from FIG. 1, wherein FIG. 9 shows the semi-rigid malleable core encompassed by the resilient outer wrap to form the planar shaped beam, wherein the resilient outer wrap has an outer surface that further comprises a first unidirectional set of angled resilient friction fingers on the beam first end portion planar primary side portion; and

FIG. 10 is cross section cut 10-10 from FIG. 1, wherein FIG. 10 shows the semi-rigid malleable core encompassed by the resilient outer wrap to form the planar shaped beam, wherein the resilient outer wrap has the outer surface that further comprises a second unidirectional set of angled resilient friction fingers on the beam second end portion planar primary side portion.

### DETAILED DESCRIPTION OF THE INVENTION

50 Support apparatus for an individual’s 55 head 60 forward flexion movement 65 to be limited

55 Individual or user

60 Head

65 Forward head 60 flexion movement limitation

70 Chin of the individual 55 head 60

75 Forward lower jaw portion

80 Manubrium

85 Sternum

90 Planar shape beam

95 First end portion of the planar shape beam 90

100 Second end portion of the planar shape beam 90

105 Mid-portion of the planar shape beam 90

110 Longitudinal axis of the planar shape beam 90

115 Crosswise axis of the planar shape beam 90

120 Perpendicular position of the cross wise axis 115 to the longitudinal axis 110

125 Parallel position of the cross wise axis 115 to the planar portion 130 of the beam 90

130 Planar shape portion of the beam 90

135 Shortwise axis of the beam 90

140 Perpendicular position of the shortwise axis 135 to the longitudinal axis 110 and the planar portion 130 of the beam 90

145 Primary side portion of the planar shape 130

150 Secondary side portion of the planar shape 130

155 Primary concave arcuate shape

160 Parallel position of the primary concave arcuate shape 155 to the crosswise axis 115

165 Open primary channel

170 Position along the longitudinal axis 110 of the open primary channel 165 from the first end portion 95, the to the mid portion 105, to the opposing second end portion 100

175 Secondary concave arcuate shape

180 Positioned parallel to the longitudinal axis 110 of the secondary concave arcuate 175 encompassing the mid-portion 105 of the beam 90

185 Cradle of the chin 70 and forward lower jaw 75 of the open primary channel 165 at the beam 90 first end portion 95 on the planar primary side portion 145

190 Adapted to rest against the manubrium 80 by the open primary channel 165 at the beam 90 second end portion 100 on the planar primary side portion 145

195 Semi-rigid malleable core

200 Resilient outer wrap that encompasses the semi-rigid malleable core 195

205 Outer surface of the resilient outer wrap 200

210 First unidirectional set of angled resilient friction fingers disposed on the beam 90 first end portion 95 on the planar primary side portion 145

211 Acute angle of the first unidirectional set of angled resilient friction fingers 210 to the longitudinal axis 110

215 Higher friction of the first unidirectional set of angled resilient friction fingers 210 along the longitudinal axis 110 from the first end portion 95 to the mid-portion 105

220 Lower friction of the first unidirectional set of angled resilient friction fingers 210 along the longitudinal axis 110 from the mid-portion 105 to the first end portion 95

225 Second unidirectional set of angled resilient friction fingers disposed on the beam 90 second end portion 100 on the planar primary side portion 145

226 Acute angle of the second unidirectional set of angled resilient friction fingers 225 to the longitudinal axis 110

230 Higher friction of the second unidirectional set of angled resilient friction fingers 225 along the longitudinal axis 110 from the second end portion 100 to the mid-portion 105

235 Lower friction of the second unidirectional set of angled resilient friction fingers 225 along the longitudinal axis 110 from the mid-portion 105 to the second end portion 100

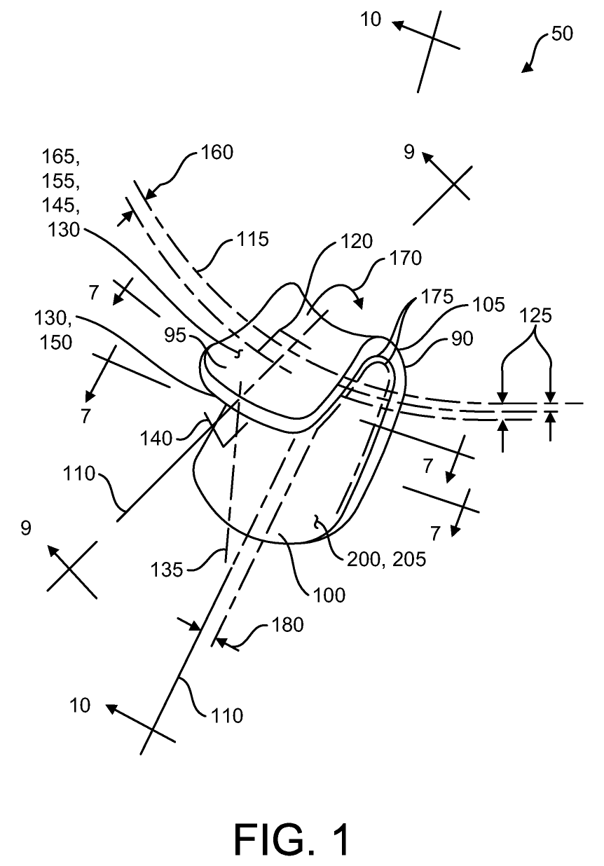

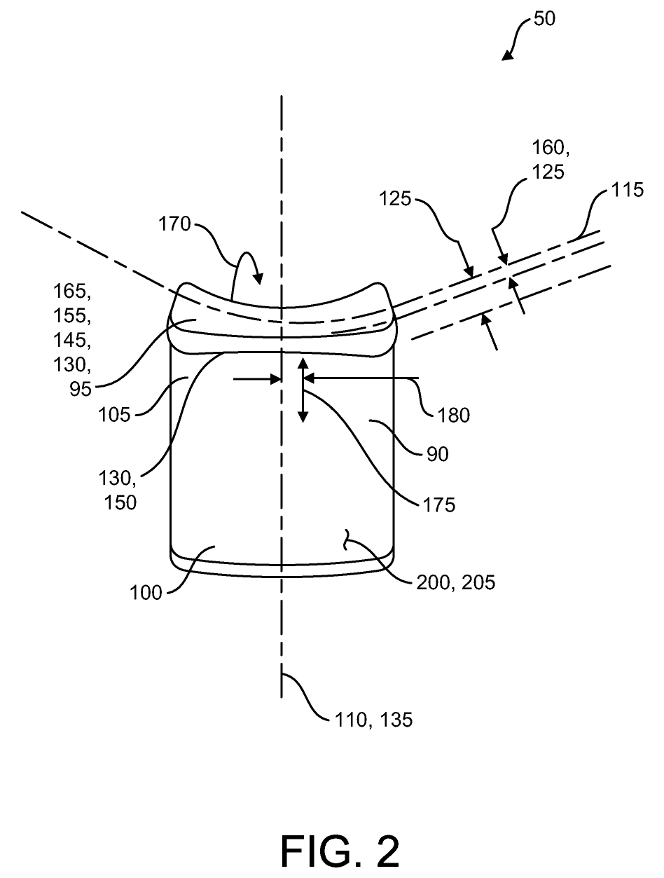

With initial reference to FIG. 1 shown is an upper perspective view of the perspective view of the individual’s 55 head 60 forward flexion movement 65 support apparatus 50 that includes the planar shaped beam 90 that further has the first end portion 95, the mid portion 105, and the second end portion 100, with the longitudinal axis 110 spanning therebetween, also with the primary 155 and secondary 175 concave arcuate shapes shown. Continuing, FIG. 2 is a front elevation view of the individual’s 55 head 60 forward flexion movement 65 support apparatus 50 that includes the planar shaped beam 90 that further has the first end portion 95, the mid portion 105, and the second end portion 100, with the longitudinal axis 110 spanning therebetween, also with the primary concave arcuate shape 155 shown.

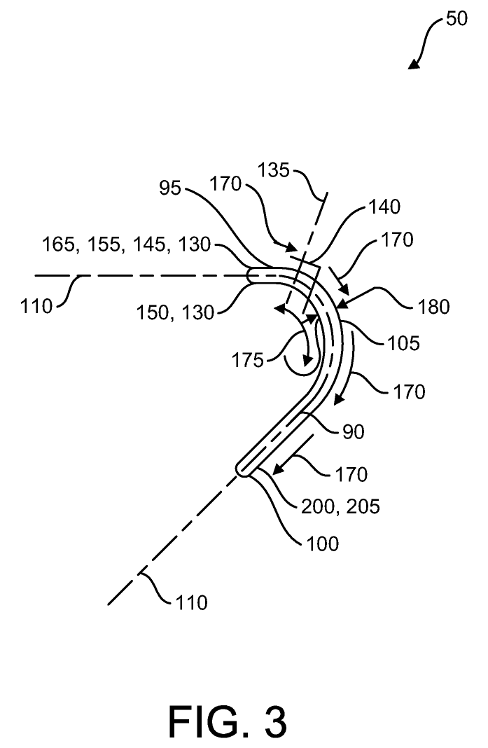

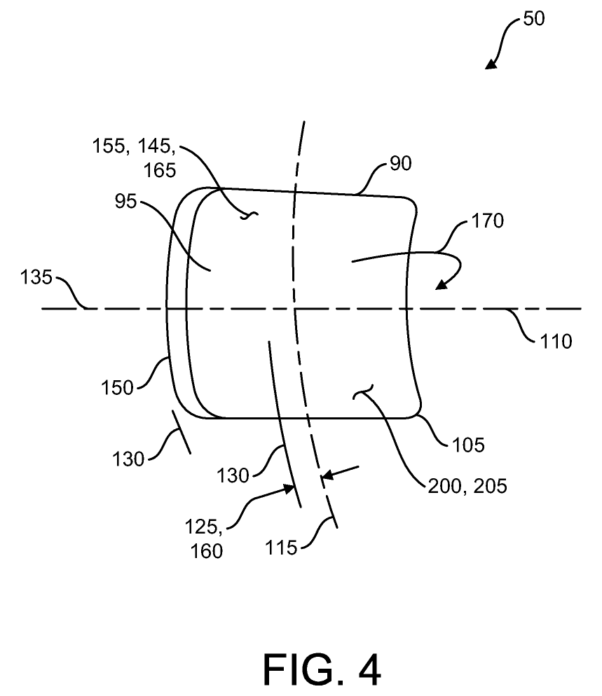

Further, FIG. 3 is a side elevation view of the individual’s 55 head 60 forward flexion movement 65 support apparatus 50 that includes the planar shaped beam 90 that further has the first end portion 95, the mid portion 105, and the second end portion 100, with the longitudinal axis 110 spanning therebetween, also with the secondary concave arcuate shape 175 shown. Next, FIG. 4 is a top view of the individual’s 55 head 60 forward flexion movement 65 support apparatus 50 that includes the planar shaped beam 90 that further has the first end portion 95, the mid portion 105, and the second end portion 100, with the longitudinal axis 110 spanning therebetween, also with the primary concave arcuate shape 155 shown.

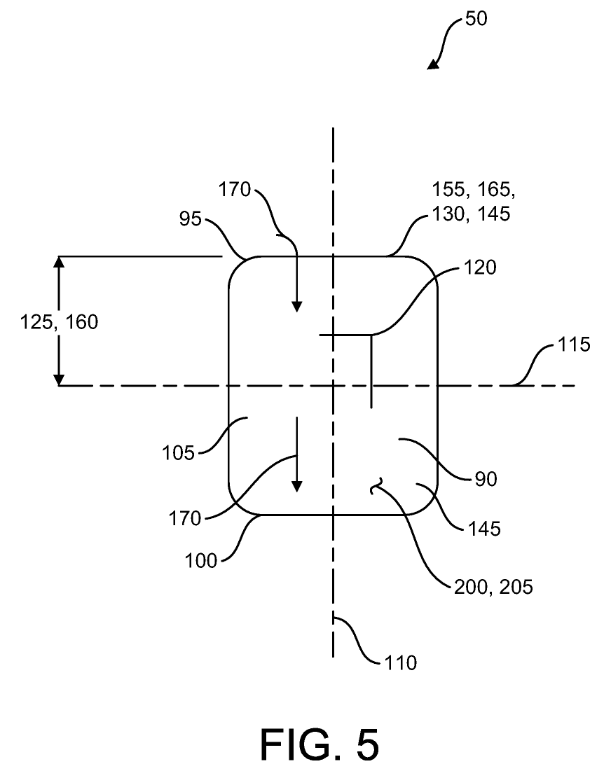

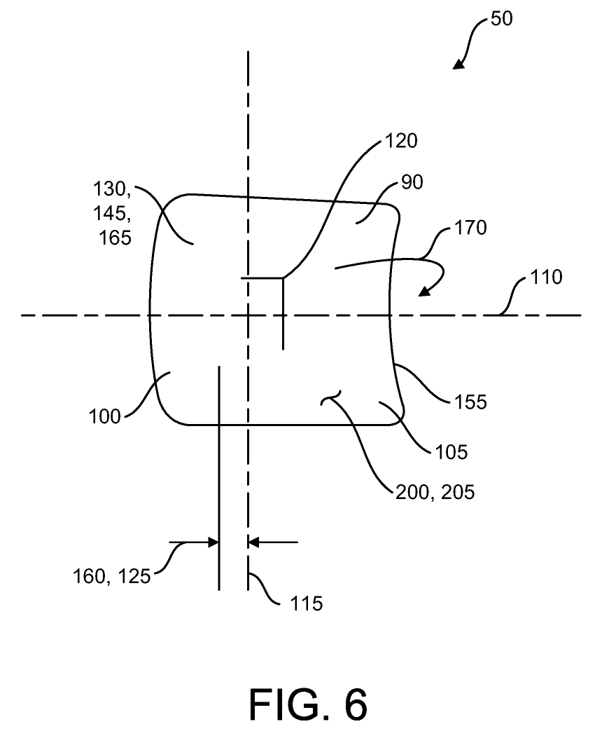

Moving onward, FIG. 5 is a rear view of the individual’s 55 head 60 forward flexion movement 65 support apparatus 50 that includes the planar shaped beam 90 that further has the first end portion 95, the mid portion 105, and the second end portion 100, with the longitudinal axis 110 spanning therebetween. Also, FIG. 6 is a bottom view of the individual’s 55 head 60 forward flexion movement 65 support apparatus 50 that includes the planar shaped beam 90 that further has the first end portion 95, the mid portion, 105 and the second end portion 100, with the longitudinal axis 110 spanning therebetween, also with the primary concave arcuate shape 155 shown.

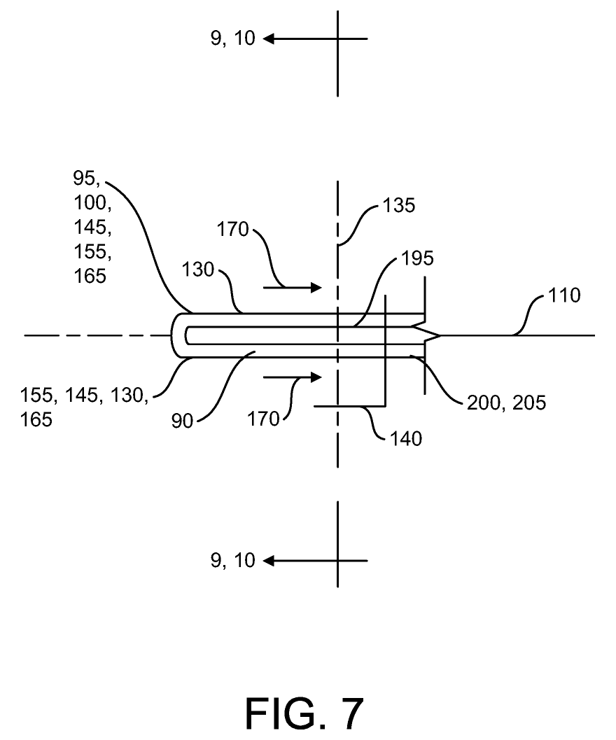

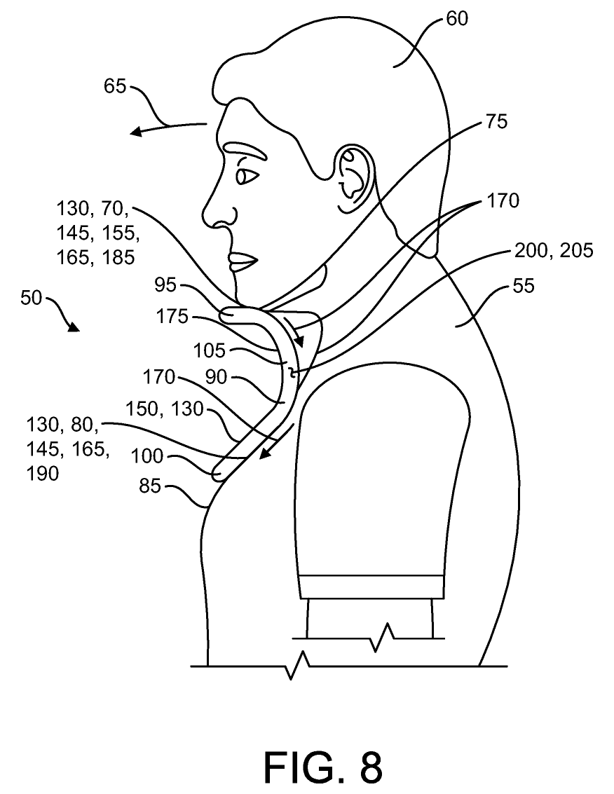

Continuing, FIG. 7 is cross section cut 7-7 positioned in two places from FIG. 1, wherein FIG. 7 of the individual’s 55 head 60 forward flexion movement 65 support apparatus 50 shows a semi-rigid malleable core 195 encompassed by a resilient outer wrap 200 to form the planar shaped beam 90. Further, FIG. 8 is an in use view shown as a side elevation view of the individual’s 55 head 60 forward flexion movement 65 support apparatus 50 that includes the planar shaped beam 90 that further has the first end portion 95, the mid portion 105, and the second end portion 100, with the longitudinal axis 110 spanning therebetween, also with the secondary concave arcuate shape 175 shown, wherein the second end portion 100 of the planar beam 90 open primary channel 165 that is adapted to rest 190 against the manubrium 80 of the individual’s 55 sternum 85 and further the first end portion 95 of the planar beam 90 that through the open primary channel 165 cradles the chin 70 and forward lower jaw 75 of the individual 55 providing head forward movement 65 limitation flexion support relative to the individual’s 55 sternum 85.

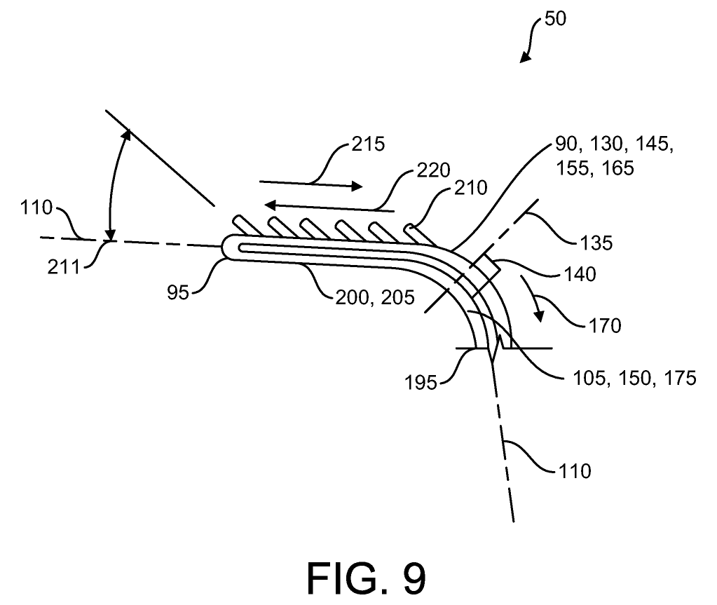

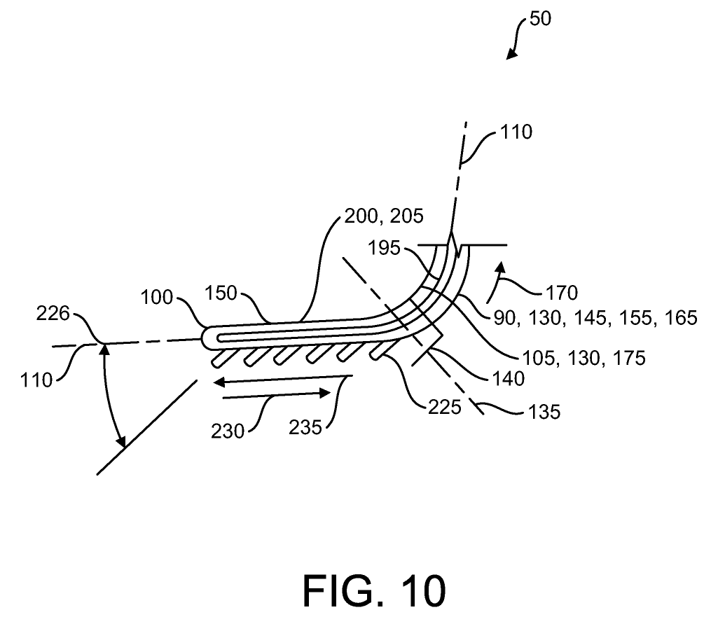

Next, FIG. 9 is cross section cut 9-9 from FIG. 1, wherein FIG. 9 of the individual’s 55 head 60 forward flexion movement 65 support apparatus 50 shows the semi-rigid malleable core 195 encompassed by the resilient outer wrap 200 to form the planar shaped beam 90, wherein the resilient outer wrap 200 has an outer surface 205 that further comprises a first unidirectional set of angled resilient friction fingers 210 on the beam 90 first end portion 95 planar primary side portion 145. Following, FIG. 10 is cross section cut 10-10 from FIG. 1, wherein FIG. 10 shows the individual’s 55 head 60 forward flexion movement 65 support apparatus 50 that shows the semi-rigid malleable core 195 encompassed by the resilient outer wrap 200 to form the planar shaped beam 90, wherein the resilient outer wrap 200 has the outer surface 205 that further comprises a second unidirectional set of angled resilient friction fingers 225 on the beam 90 second end portion 100 planar primary side portion 145.

Broadly, in looking at FIGS. 1 to 10, the present invention is the individual’s 55 head 60 forward flexion movement 65 support apparatus 50, wherein the individual’s 55 head 60 includes a chin 70 and a forward lower jaw portion 75, the individual 55 further having a manubrium portion 80 of a sternum 85, the support apparatus 50 includes the planar shape beam 90 having the first end portion 95 and the opposing second end portion 100, with the beam mid-portion 105 disposed therebetween the beam first 95 and second 100 end portions. The beam 90 having the longitudinal axis 110 spanning therebetween the beam first 95 and second 100 end portions, further the beam 90 including a crosswise axis 115 positioned perpendicular 120 to the longitudinal axis 110, the crosswise axis 115 is also parallel 125 to the planar portion 130 of the beam 90, also a shortwise axis 135 is positioned perpendicular 140 to both the longitudinal axis 110 and the planar portion 130 of the beam 90.

The individual’s 55 head 60 forward flexion movement 65 support apparatus 50 planar shape 130 having the primary side portion 145 and the opposing secondary side portion 150, the planar shape 130 primary side portion 145 having a primary concave arcuate shape 155 parallel 160 to the crosswise axis 115, the primary concave arcuate shape 155 forming the open primary channel 165 along the longitudinal axis 110 from the first end portion 95 to the beam 90 mid-portion 105 to the beam 90 opposing second end portion 100. The planar shape 130 secondary side portion 150 having the secondary concave arcuate shape 175 parallel 180 to the longitudinal axis 110 encompassing the beam 90 mid-portion 105.

Wherein operationally, the beam 90 first end portion 95 planar 130 primary side portion 145 open primary channel 155 is adapted 185 to cradle the chin 70 and forward lower jaw 75 of the users 55 head 60, the beam 90 second end portion 100 planar 130 primary side portion 145 open primary channel 155 is adapted 190 to rest against the manubrium 80 resulting in the support apparatus 50 supporting the individual’s 55 head 60 to limit forward flexion head movement 65 in relation to the manubrium 80, see in particular FIG. 8.

As an alternative to the individual’s 55 head 60 forward flexion movement 65 support apparatus 50, is wherein the planar shape 130 beam 90 can be constructed of a semi-rigid malleable core 195 to operationally give the planar shape 130 beam 90 individual 55 customizable bendable rigidity to fit the individual 55 better for the particular positional relationship between their manubrium 80 and their forward lower jaw 75 with chin 70 to form a selected shape of the beam 90, see in particular FIGS. 7, 8, 9, and 10.

As a further alternative to the individual’s 55 head 60 forward flexion movement 65 support apparatus 50, it can further comprise a resilient outer wrap 200 that encompasses the semi-ridged malleable core 195 to operationally provide a higher friction outer surface 205 for the individual’s 55 chin 70, lower forward jaw 75, and manubrium 80, see in particular FIGS. 7, 8, 9, and 10.

Continuing as an option for the individual’s 55 head 60 forward flexion movement 65 support apparatus 50, wherein the resilient outer wrap 200 has the outer surface 205 that can include the first unidirectional set of angled resilient friction fingers 210 on the beam 90 first end portion 95 planar 130 primary side portion 145 that have higher friction 215 along the longitudinal axis 110 from the beam 90 first end portion 95 to the beam 90 mid-portion 105 than a lower friction 220 in an opposing direction along the longitudinal axis 110 from the beam 90 mid portion 105 to the beam 90 first end portion 95, to operationally help hold the planar shaped 130 beam 90 upon the individual’s 55 chin 70 and forward lower jaw 75, see in particular FIGS. 8 and 9.

A further option for the individual’s 55 head 60 forward flexion movement 65 support apparatus 50, is wherein the resilient outer wrap 200 has the outer surface 205 that can include the second unidirectional set of angled resilient friction fingers 225 on the beam 90 second end portion 100 planar 130 primary side portion 145 that have higher friction 230 along the longitudinal axis 110 from the beam 90 second end portion 100 to the beam 90 mid-portion 105 than a lower friction 235 in an opposing direction along the longitudinal axis 110 from the beam 90 mid portion 105 to the beam 90 second end portion 100 to operationally help hold the planar shaped 130 beam 90 upon the individual’s 55 manubrium 80, see in particular FIGS. 8 and 10.

Accordingly, the present invention of the individual’s head forward flexion movement support apparatus has been described with some degree of particularity directed to the embodiments of the present invention. It should be appreciated, though; that the present invention is defined by the following claims construed in light of the prior art so modifications of the changes may be made to the exemplary embodiments of the present invention without departing from the inventive concepts contained therein.

1. A support apparatus configured for supporting an individual's head to limit a forward flexion head movement in relation to a manubrium, said support apparatus comprises:
a. a planar shape beam having a first end portion and an opposing second end portion, with a planar shape beam mid-portion disposed therebetween said beam first and second end portions, said first end portion and said opposing second end portion said planar shape beam having a longitudinal axis spanning therebetween said planar shape beam first and second end portions, further said planar shape beam including a crosswise axis positioned perpendicular to said longitudinal axis, said crosswise axis is also parallel to said a planar portion of said planar shape beam, also a shortwise axis is positioned perpendicular to both said longitudinal axis and said planar portion of said planar shape beam, said planar shape beam having a primary side portion and an opposing secondary side portion, said planar shape primary side portion said primary side portion having a primary concave arcuate shape parallel to said crosswise axis, said primary concave arcuate shape forming an open primary channel along said longitudinal axis from said
first end portion to said planar shape beam mid-portion and onto , said opposing second end portion,said opposing secondary side portion having a secondary concave arcuate shape parallel to said longitudinal axis encompassing said planar shape beam mid-portion, wherein said first end portion, said mid-portion, and said second end portion combined form a "U" shape alonq said lonqitudinal axis, wherein operationally said open primary channel is adapted to cradle the chin and forward lower jaw of the users head, said open primary channel is adapted to solely rest against the manubrium resulting in said support apparatus supporting the individual's head to limit forward flexion head movement in relation to the manubrium, with said first end portion and said second end portion facing one another in compression to one another resulting in bending of said mid-portion as between only the chin and forward lower jaw as against the manubrium, wherein said planar shape beam is constructed of a semi-riqid malleable core to operationally give said planar shape beam individual customizable bendable rigidity to form a selected shape.

2. (canceled)

3. The support apparatus according to claim 1 further comprising a resilient outer wrap that encompasses said semi-rigid malleable core confiqured to provide a higher friction surface for the individual's chin, lower forward jaw, and manubrium.

4. The support apparatus according to claim 3 wherein said resilient outer wrap has an outer surface that includes a first unidirectional set of fixed acute angled resilient friction fingers each with a rounded smooth tip on said primary side portion that have higher friction along said longitudinal axis from said first end portion to said beam mid-portion and a lower friction in an opposing direction along said longitudinal axis from said beam mid portion to said beam first end portionconfigured to help hold said planar shape beam upon the individual's chin and forward lower jaw.

5. The support apparatus according to claim 4 wherein said resilient outer wrap has an outer surface that includes a second unidirectional set of fixed acute angled resilient friction fingers each with a rounded smoothtip on said primary side portion that have higher friction along said longitudinal axis from said opposinq second end portion to said beam mid-portion and a lower friction in an opposing direction along said longitudinal axis from said beam mid portion to said beam second end portion configured to help hold said planar shape beam upon the individual's manubrium.
